(19) United States
(12) Patent Application Publication
(10) Pub. No.: US 2008/0248952 A1
(43) Pub. Date: Oct. 9, 2008

(54) Substituted 6-Phenyl-7-Aminotriazolopyrimidines, Method for the Production Thereof, Their Use for Controlling Pathogenic Fungi, and Agents Containing These Compounds

(75) Inventors: Carsten Blettner, Hong Kong (CN); Markus Gewehr, Kastellaun (DE); Wassilios Grammenos, Ludwigshafen (DE); Thomas Grote, Wachenheim (DE); Udo Hunger, Mainz (DE); Bernd Muller, Frankenthal (DE); Matthias Niedenbruck, Limburgerhof (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Barbara Nave, Deidesheim (DE); Maria Scherer, Godramstein (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 11/631,004

(22) PCT Filed: Jul. 6, 2005
(86) PCT No.: PCT/EP2005/007277

§ 371 Date: Dec. 28, 2006

(30) Foreign Application Priority Data
Jul. 8, 2004 (DE) 10 2004 033 239.8

(51) Int. Cl. C07D 487/04; A01N 43/90; A01N 25/00

(52) U.S. Cl. 504/100; 544/263; 514/259.31

(57) ABSTRACT

Substituted triazolopyrimidines of the formula I

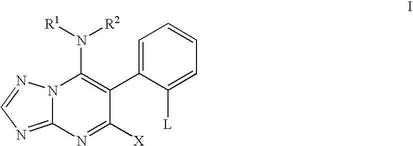

in which the substituents are as defined below:
R¹ is alkyl, haloalkyl, cycloalkyl, halocycloalkyl, alkenyl, haloalkenyl, cycloalkenyl, halocycloalkenyl, alkynyl, haloalkynyl or phenyl, naphthyl, or a five- or six-membered saturated, partially unsaturated or aromatic heterocycle comprising one to four heteroatoms from the group consisting of O, N and S,
R² is hydrogen or a group R¹,
   R¹ and R² together with the nitrogen atom, to which they are attached, may also form a five- or six-membered heterocyclyl or heteroaryl which is attached via N and which may comprise one to three further heteroatoms from the groups consisting of O, N and S as ring member and/or may be substituted according to the description;
L is fluorine, chlorine or methyl;
X is cyano, C₁-C₄-alkyl, C₁-C₄-alkoxy or C₁-C₂-haloalkoxy,
   where X is not methyl if R¹ and R² together are n-pentylene or 3-methyl-n-pentylene and L is fluorine, or R¹ and R² together are 3-methyl-n-pentylene and L is chlorine;
   processes and intermediates for preparing these compounds, compositions comprising them and their use for controlling phytopathogenic harmful fungi.

The present invention relates to substituted triazolopyrimidines of the formula I

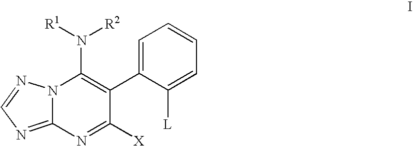

in which the substituents are as defined below:
R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle comprising one to four heteroatoms from the group consisting of O, N and S,
R² is hydrogen or one of the groups mentioned under R¹,
   R¹ and R² together with the nitrogen atom, to which they are attached; may also form a five- or six-membered heterocyclyl or heteroaryl which is attached via N and which may comprise one to three further heteroatoms from the group consisting of O, N and S as ring member and/or carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-halo-alkenyloxy, (exo)-C₁-C₆-alkylene and oxy-C₁-C₃-alkyleneoxy;
R¹ and/or R² may carry one to four identical or different groups R^{a}:
   R^{a} is halogen, cyano, nitro, hydroxyl, C₁-C₆-alkyl, C₁-C₆-haloalkyl, C₁-C₆-alkyl-carbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkyleneoxy, phenyl, naphthyl, a five- to ten-membered saturated, partially unsaturated or aromatic heterocycle comprising one to four heteroatoms from the group consisting of O, N and S,
      where these aliphatic, alicyclic or aromatic groups for their part may be partially or fully halogenated or may carry one to three groups R^{b}:
         R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals comprise 1 to 6 carbon atoms and the alkenyl or alkynyl groups mentioned in these radicals comprise 2 to 8 carbon atoms;
            and/or one to three of the following radicals:
            cycloalkyl, cycloalkoxy, heterocyclyl, heterocyclyloxy, where the cyclic systems comprise 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably comprise 6 to 10 ring members and the hetaryl radicals comprise 5 or 6 ring members, where the cyclic systems may be partially or fully halogenated or substituted by alkyl or haloalkyl groups;
L is fluorine, chlorine or methyl, and;
X is cyano, C₁-C₄-alkyl, C₁-C₄-alkoxy or C₁-C₂-haloalkoxy;
   where X is not methyl if R¹ and R² together are n-pentylene or 3-methyl-n-pentylene and L is fluorine, or R¹ and R² together are 3-methyl-n-pentylene and L is chlorine.

Moreover, the invention relates to processes and intermediates for preparing these compounds, compositions comprising them and their use for controlling phytopathogenic harmful fungi.

5-Chloro-6-phenyl-7-aminotriazolopyrimidines are known in a general manner from EP-A 71 792, EP-A 550 113. U.S. Pat. No. 5,994,360 discloses individual 5-methyl-6-phenyl-7-aminotriazolopyrimidines, JP-A 2002-308879 proposes, in a general manner, 5-haloalkyl-6-phenyl-7-aminotriazolopyrimidines. It is known that these compounds are suitable for controlling harmful fungi.

The compounds according to the invention differ from those described in the above-mentioned publications by the specific combination of the ortho-substituted 6-phenyl group with the substituents in the 5- and 7-positions of the triazolopyrimidine skeleton.

However, the fungicidal action of the prior-art compounds is in many cases unsatisfactory. Accordingly, it is an object of the present invention to provide compounds having improved activity and/or a broader activity spectrum.

We have found that this object is achieved by the compounds defined at the outset. Furthermore, we have found processes and intermediates for their preparation, compositions comprising them and methods for controlling harmful fungi using the compounds I.

The novel compounds according to the invention can be obtained by different routes.

The novel compounds according to the invention can be obtained by different routes. Advantageously, they are prepared by reacting 5-aminotriazole of the formula II with appropriately substituted phenylmalonates of the formula III in which R is alkyl, preferably C₁-C₆-alkyl, in particular methyl or ethyl.

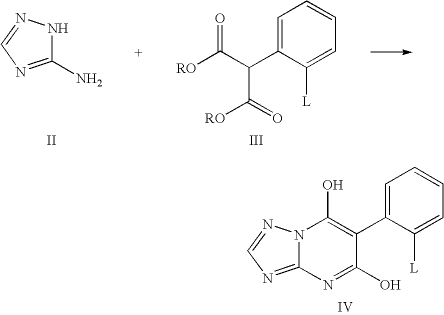

This reaction is usually carried out at temperatures of from 80° C. to 250° C., preferably from 120° C. to 180° C., in the absence of a solvent or in an inert organic solvent in the presence of a base [cf. EP-A 770 615] or in the presence of acetic acid under the conditions known from Adv. Het. Chem. 57 (1993), 81ff.

Suitable solvents are aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethyl-acetamide. The reaction is particularly preferably carried out in the absence of a solvent or in chlorobenzene, xylene, dimethyl sulfoxide or N-methylpyrrolidone. It is also possible to use mixtures of the solvents mentioned.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates, and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to tertiary amines such as triisopropylethylamine, tributylamine, N-methylmorpholine or N-methylpiperidine.

The bases are generally employed in catalytic amounts; however, they can also be employed in equimolar amounts, in excess or, if appropriate, as solvents.

The starting materials are generally reacted with one another in equimolar amounts. In terms of yield, it may be advantageous to employ an excess of base and malonate III, based on the triazole.

Phenylmalonates of the formula III are advantageously obtained by reacting appropriately substituted bromobenzenes with dialkyl malonates under Cu(I) catalysis [cf. Chemistry Letters (1981), 367-370; EP-A 10 02 788].

The dihydroxytriazolopyrimidines of the formula IV are converted under the conditions known from WO-A 94/20501 into the dihalopyrimidines of the formula V, in which Hal is a halogen atom, preferably a bromine or a chlorine atom, in particular a chlorine atom. Advantageous halogenating agents [HAL] are chlorinating agents or brominating agents, such as phosphorus oxybromide or phosphorus oxychloride, if appropriate in the presence of a solvent.

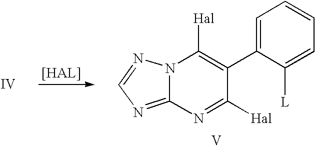

This reaction is usually carried out at from 0° C. to 150° C., preferably at from 80° C. to 125° C. [cf. EP-A 770 615].

Dihalopyrimidines of the formula V are reacted further with amines of the formula VI

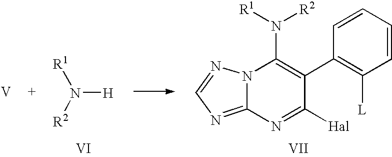

in which R¹ and R² are as defined in formula I to give 5-halotriazolopyrimines of the formula VII.

This reaction is advantageously carried out at from 0° C. to 70° C., preferably from 10° C. to 35° C., preferably in the presence of an invert solvent, such as an ether, for example dioxane, diethyl ether or, in particular, tetrahydrofuran, a halogenated hydrocarbon, such as dichloromethane, or an aromatic hydrocarbon, such as, for example, toluene [cf. WO-A 98/46608].

The use of a base, such as a tertiary amine, for example triethylamine, or an inorganic amine, such as potassium carbonate, is preferred; it is also possible for excess amine of the formula VI to serve as base.

Compounds of the formula I in which X is cyano, C₁-C₆-alkoxy or C₁-C₂-haloalkoxy can be obtained in an advantageous manner by reacting compounds I in which X is halogen, preferably chlorine, with compounds M-X′ (formula VIII). Depending on the meaning of the group X′ to be introduced, the compounds VII are inorganic cyanides, alkoxides or haloalkoxides. The reaction is advantageously carried out in the presence of an inert solvent. The cation M in formula VIII is of little importance; for practical reasons, ammonium, tetraalkylammonium or alkali metal or alkaline earth metal salts are usually preferred.

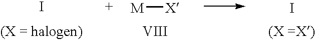

The reaction temperature is usually from 0 to 120° C., preferably from 10 to 40° C. [cf. J. Heterocycl. Chem. 12 (1975), 861-863].

Suitable solvents include ethers, such as dioxane, diethyl ether and, preferably, tetrahydrofuran, halogenated hydrocarbons, such as dichloromethane, and aromatic hydrocarbons, such as toluene.

Compounds of the formula I, in which X is C₁-C₄-alkyl or can be obtained in an advantageous manner by the following synthesis route:

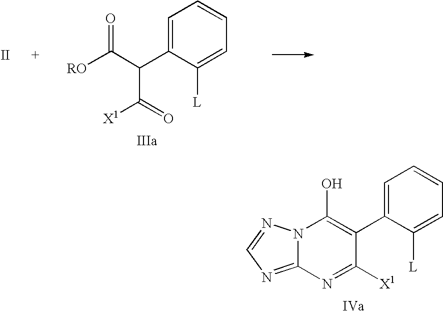

Starting with the keto esters IIIa, the 5-alkyl-7-hydroxy-6-phenyltriazolopyrimidines IVa are obtained. In the formula IIIa and IVa, X¹ is C₁-C₄-alkyl or C₁-C₄-haloalkyl. By using the easily obtainable 2-phenylacetoacetates (IIIa where X¹═CH₃), the 5-methyl-7-hydroxy-6-phenyltriazolopyrimidines are obtained [cf. Chem. Pharm. Bull. 9 (1961), 801]. The starting materials IIIa are advantageously prepared under the conditions described in EP-A 10 02 788.

The resulting 5-alkyl-7-hydroxy-6-phenyltriazolopyrimidines are reacted with halogenating agents [HAL] under the conditions described further above to give the 7-halotriazolopyrimidines of the formula Va. Preference is given to using chlorinating or brominating agents, such as phosphorus oxybromide, phosphorus oxychloride, thionyl chloride, thionyl bromide or sulfuryl chloride. The reaction can be carried out neat or in the presence of a solvent. Customary reaction temperatures are from 0 to 150° C. or, preferably, from 80 to 125° C.

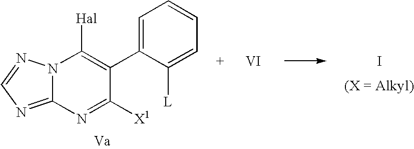

The reaction of Va with amines VI is carried out under the conditions described further above.

Alternatively, compounds of the formula I in which X is C₁-C₄-alkyl can also be prepared from compounds VII in which X is halogen, in particular chlorine, and malonates of the formula IX. In formula IX, X″ is hydrogen or C₁-C₃-alkyl and R is C₁-C₄-alkyl. They are converted into compounds of the formula X and decarboxylated to give compounds I [cf. U.S. Pat. No. 5,994,360].

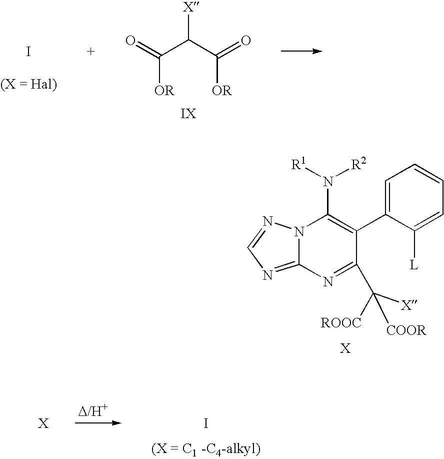

The malonates IX are known from the literature [J. Am. Chem. Soc. 64 (1942), 2714; J. Org. Chem. 39 (1974), 2172; Helv. Chim. Acta, 61 (1978), 1565], or they can be prepared in accordance with the literature cited.

The subsequent hydrolysis of the ester X is carried out under generally customary conditions; depending on the various structural elements, the alkaline or the acidic hydrolysis of the compounds X may be advantageous. Under the conditions of the ester hydrolysis, there may already be complete or partial decarboxylation to 1.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethyl-formamide and dimethylacetamide; particularly preferably, the reaction is carried out in hydrochloric acid or acetic acid. It is also possible to use mixtures of the solvents mentioned.

Compounds of the formula I in which X is C₁-C₄-alkyl can also be obtained by coupling 5-halotriazolopyrimidines of the formula VII with organometallic reagents of the formula XI. In one embodiment of this process, the reaction is carried out with transition metal catalysis, such as Ni or Pd catalysis.

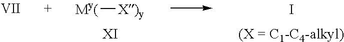

In formula XI, M is a metal ion of the valence y, such as, for example, B, Zn or Sn, and X″ is C₁-C₃-alkyl. This reaction can be carried out, for example, analogously to the following methods: J. Chem. Soc. Perkin Trans. 1 (1994), 1187, ibid 1 (1996), 2345; WO-A 99/41255; Aust. J. Chem. 43 (1990), 733; J. Org. Chem. 43 (1978), 358; J. Chem. Soc. Chem. Commun. (1979), 866; Tetrahedron Lett. 34 (1993), 8267; ibid 33 (1992), 413.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plants, or in the harmful fungus to be controlled.

In the definitions of the symbols given in the formulae above, collective terms were used which are generally representative of the following substituents:

halogen: fluorine, chlorine, bromine and iodine;
alkyl: saturated straight-chain or branched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethyl-propyl, 1,2-dimethylpropyl, 1-methylpentyl, 2-methylpentyl, 3-methylpentyl, 4-methyl-pentyl, 1,1-dimethylbutyl, 1,2-dimethylbutyl, 1,3-dimethylbutyl, 2,2-dimethylbutyl, 2,3-di-methylbutyl, 3,3-dimethylbutyl, 1-ethylbutyl, 2-ethylbutyl, 1,1,2-trimethylpropyl, 1,2,2-tri-methylpropyl, 1-ethyl-1-methylpropyl and 1-ethyl-2-methylpropyl;
haloalkyl: straight-chain or branched alkyl groups having 1 to 2, 4 or 6 carbon atoms (as mentioned above), where in these groups some or all of the hydrogen atoms may be replaced by halogen atoms as mentioned above; in particular, C₁-C₂-haloalkyl, such as chloromethyl, bromomethyl, dichloromethyl, trichloromethyl, fluoromethyl, difluoro-methyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-tri-fluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated straight-chain or branched hydrocarbon radicals having 2 to 8 carbon atoms and one or two double bonds in any position (as mentioned above), where in these groups some or all of the hydrogen atoms may be replaced by halogen atoms as mentioned above, in particular by fluorine, chlorine and bromine;
alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;
five- to ten-membered saturated, partially unsaturated or aromatic heterocycle comprising one to four heteroatoms from the group consisting of O, N and S:
5- or 6-membered heterocyclyl comprising one to three nitrogen atoms and/or one oxygen or sulfur atom or one or two oxygen and/or sulfur atoms, for example 2-tetra-hydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydro-pyrimidinyl and 2-piperazinyl;
5-membered heteroaryl comprising one to four nitrogen atoms or one to three nitrogen atoms and one sulfur or oxygen atom: 5-membered heteroaryl groups which, in addition to carbon atoms, may comprise one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl comprising one to three or one to four nitrogen atoms: 6-membered heteroaryl groups which, in addition to carbon atoms, may comprise one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;
   alkylene: divalent unbranched chains of 3 to 5 CH₂ groups, for example CH₂, CH₂CH₂, CH₂CH₂CH₂, CH₂CH₂CH₂CH₂ and CH₂CH₂CH₂CH₂CH₂;
   oxyalkylene: divalent unbranched chains of 2 to 4 CH₂ groups, where one valence is attached to the skeleton via an oxygen atom, for example OCH₂CH₂, OCH₂CH₂CH₂ and OCH₂CH₂CH₂CH₂;
   oxyalkyleneoxy: divalent unbranched chains of 1 to 3 CH₂ groups, where both valences are attached to the skeleton via an oxygen atom, for example OCH₂O, OCH₂CH₂O and OCH₂CH₂CH₂O.

The scope of the present invention includes the (R)- and (S)-isomers and the racemates of compounds of the formula I having chiral centers.

With a view to the intended use of the triazolopyrimidines of the formula I, particular preference is given to the following meanings of the substituents, in each case on their own or in combination:

Preference is given to compounds of the formula I in which R¹ is not hydrogen.

Particular preference is given to compounds I in which R¹ is C₁-C₆-alkyl,

C₂-C₆-alkenyl or C₁-C₈-haloalkyl.

Preference is given to compounds I in which R¹ is a group A:

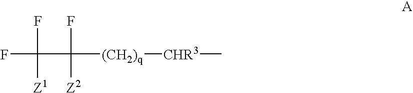

in which
Z¹ is hydrogen, fluorine or C₁-C₆-fluoroalkyl,
Z² is hydrogen or fluorine, or
   Z¹ and Z² together form a double bond;
q is 0 or 1; and
R³ is hydrogen or methyl.

Moreover, preference is given to compounds I in which R¹ is C₃-C₆-cycloalkyl which may be substituted by C₁-C₄-alkyl.

Particular preference is given to compounds I in which R² is hydrogen.

Preference is likewise given to compounds I in which R² is methyl or ethyl.

If R¹ and/or R² comprise haloalkyl or haloalkenyl groups having a center of chirality, the (S) isomers are preferred for these groups. In the case of halogen-free alkyl or alkenyl groups having a center of chirality in R¹ or R², preference is given to the (R) configured isomers.

Preference is furthermore given to compounds I in which R¹ and R² together with the nitrogen atom to which they are attached form a piperidinyl, morpholinyl or thiomorpholinyl ring, in particular a piperidinyl ring, which, if appropriate, is substituted by one to three halogen, C₁-C₄-alkyl or C₁-C₄-haloalkyl groups. Particularly preference is given to the compounds in which R¹ and R² together with the nitrogen atom to which they are attached form a 2-methyl-, 3-methyl- or 3,5-dimethylpiperidine ring.

In addition, preference is given to compounds I in which R¹ and R² together with the nitrogen atom to which they are attached form one of the abovementioned five-membered saturated or unsaturated rings or a morpholinyl or thiomorpholinyl ring, where the rings may be substituted by one to three halogen, C₁-C₄-alkyl or C₁-C₄-haloalkyl groups.

The invention particularly preferably provides compounds I in which R¹ and R² together with the nitrogen atom to which they are attached form a pyrazole or pyrrolidine ring which may be substituted by one or two halogen, C₁-C₄-alkyl or C₁-C₄-haloalkyl groups, in particular 3,5-dimethylpyrazole, 3,5-di(trifluoromethyl)pyrazole, 2-methylpyrrolidine or 3-methylpyrrolidine.

In addition, preference is also given to compounds of the formula I in which R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl; R² is hydrogen or methyl; or R¹ and R² together are —(CH₂)₂CH(CH₃)(CH₂)₂—, —CH(CH₃)(CH₂)₄—, —(CH₂)₂CH(CF₃)(CH₂)₂— or —(CH₂)₂O(CH₂)₂—.

Preference is given to compounds I in which X is C₁-C₄-alkyl, cyano or C₁-C₄-alkoxy, such as methyl, cyano, methoxy or ethoxy, especially methyl.

A preferred embodiment of the invention relates to compounds of the formula I in which L is methyl.

A further preferred embodiment of the invention relates to compounds of the formula I in which L is chlorine.

A further preferred embodiment of the invention relates to compounds of the formula I which correspond to the formula I.1:

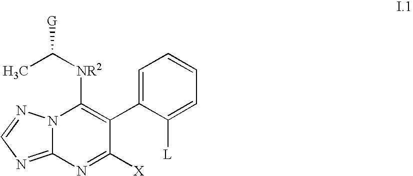

in which
G is C₂-C₆-alkyl, in particular ethyl, n- and isopropyl, n-, sec-, tert-butyl, and C₁-C₄-alkoxymethyl, in particular ethoxymethyl, or C₃-C₆-cycloalkyl, in particular cyclopentyl or cyclohexyl;
R² is hydrogen or methyl; and
X is methyl, cyano, methoxy or ethoxy.

A further preferred embodiment of the invention relates to compounds of the formula I in which R¹ and R² together with the nitrogen atom to which they are attached form a five- or six-membered heterocyclyl or heteroaryl which is attached via N and may comprise a further heteroatom from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkyleneoxy, where X is not methyl if D is n-pentylene or 3-methyl-n-pentylene and L is fluorine, or D is 3-methyl-n-pentylene and L is chlorine. These compounds correspond in particular to the formula I.2

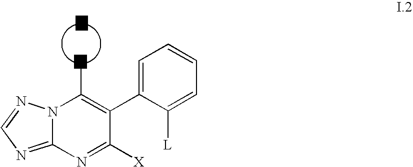

in which
D together with the nitrogen atom form a five- or six-membered heterocyclyl or heteroaryl which is attached via N and may comprise a further heteroatom from the group consisting of O, N and S as ring member and/or may carry one or more substituents from the group consisting of halogen, C₁-C₄-alkyl, C₁-C₄-alkoxy and C₁-C₂-haloalkyl; and
X is methyl, cyano, methoxy or ethoxy,
   where X is not methyl if D is n-pentylene or 3-methyl-n-pentylene and L is fluorine, or D is 3-methyl-n-pentylene and L is chlorine.

A further preferred embodiment of the invention relates to compounds of the formula I which corresponds to the formula I.3.

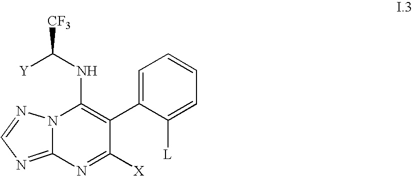

in which Y is hydrogen or C₁-C₄-alkyl, in particular methyl and ethyl, and X is methyl, cyano, methoxy or ethoxy.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

### Table 1

Compounds of the formula I in which X is cyano, L is chlorine and the combination of R¹ and R² corresponds for each compound to one row of table A

### Table 2

Compounds of the formula I in which X is methyl, L chlorine and the combination of R¹ and R² corresponds for each compound to one of the rows A-1 to A-125 of table A

### Table 3

Compounds of the formula I in which X is methoxy, L is chlorine and the combination of R¹ and R² corresponds for each compound to one row of table A

### Table 4

Compounds of the formula I in which X is cyano, L is fluorine and the combination of R¹ and R² corresponds for each compound to one row of table A

### Table 5

Compounds of the formula I in which X is methyl, L is fluorine and the combination of R¹ and R² corresponds for each compound to one of the rows A-1 to A-124 of table A

### Table 6

Compounds of the formula I in which X is methoxy, L is fluorine and the combination of R¹ and R² corresponds for each compound to one row of table A

### Table 7

Compounds of the formula I in which X is cyano, L is methyl and the combination of R¹ and R² corresponds for each compound to one row of table A

### Table 8

Compounds of the formula I in which X is methyl, L is methyl and the combination of R¹ and R² corresponds for each compound to one row of table A

### Table 9

Compounds of the formula I in which X is methoxy, L is methyl and the combination of R¹ and R² corresponds for each compound to one row of table A

TABLE A

| No. | R¹ | R² |
|---|---|---|
| A-1 | H | H |
| A-2 | CH₃ | H |
| A-3 | CH₃ | CH₃ |
| A-4 | CH₂CH₃ | H |
| A-5 | CH₂CH₃ | CH₃ |
| A-6 | CH₂CH₃ | CH₂CH₃ |
| A-7 | CH₂CF₃ | H |
| A-8 | CH₂CF₃ | CH₃ |
| A-9 | CH₂CF₃ | CH₂CH₃ |
| A-10 | CH₂CCl₃ | H |
| A-11 | CH₂CCl₃ | CH₃ |
| A-12 | CH₂CCl₃ | CH₂CH₃ |
| A-13 | CH₂CH₂CH₃ | H |
| A-14 | CH₂CH₂CH₃ | CH₃ |
| A-15 | CH₂CH₂CH₃ | CH₂CH₃ |
| A-16 | CH₂CH₂CH₃ | CH₂CH₂CH₃ |
| A-17 | CH(CH₃)₂ | H |
| A-18 | CH(CH₃)₂ | CH₃ |
| A-19 | CH(CH₃)₂ | CH₂CH₃ |
| A-20 | CH₂CH₂CH₂CH₃ | H |
| A-21 | CH₂CH₂CH₂CH₃ | CH₃ |
| A-22 | CH₂CH₂CH₂CH₃ | CH₂CH₃ |
| A-23 | CH₂CH₂CH₂CH₃ | CH₂CH₂CH₃ |
| A-24 | CH₂CH₂CH₂CH₃ | CH₂CH₂CH₂CH₃ |
| A-25 | (±)CH(CH₃)—CH₂CH₃ | H |
| A-26 | (±)CH(CH₃)—CH₂CH₃ | CH₃ |
| A-27 | (±)CH(CH₃)—CH₂CH₃ | CH₂CH₃ |
| A-28 | (S)CH(CH₃)—CH₂CH₃ | H |
| A-29 | (S)CH(CH₃)—CH₂CH₃ | CH₃ |
| A-30 | (S)CH(CH₃)—CH₂CH₃ | CH₂CH₃ |
| A-31 | (R)CH(CH₃)—CH₂CH₃ | H |
| A-32 | (R)CH(CH₃)—CH₂CH₃ | CH₃ |
| A-33 | (R)CH(CH₃)—CH₂CH₃ | CH₂CH₃ |
| A-34 | (±)CH(CH₃)—CH(CH₃)₂ | H |
| A-35 | (±)CH(CH₃)—CH(CH₃)₂ | CH₃ |
| A-36 | (±)CH(CH₃)—CH(CH₃)₂ | CH₂CH₃ |
| A-37 | (S)CH(CH₃)—CH(CH₃)₂ | H |
| A-38 | (S)CH(CH₃)—CH(CH₃)₂ | CH₃ |
| A-39 | (S)CH(CH₃)—CH(CH₃)₂ | CH₂CH₃ |
| A-40 | (R)CH(CH₃)—CH(CH₃)₂ | H |
| A-41 | (R)CH(CH₃)—CH(CH₃)₂ | CH₃ |
| A-42 | (R)CH(CH₃)—CH(CH₃)₂ | CH₂CH₃ |
| A-43 | (±)CH(CH₃)—C(CH₃)₃ | H |
| A-44 | (±)CH(CH₃)—C(CH₃)₃ | CH₃ |
| A-45 | (±)CH(CH₃)—C(CH₃)₃ | CH₂CH₃ |
| A-46 | (S)CH(CH₃)—C(CH₃)₃ | H |
| A-47 | (S)CH(CH₃)—C(CH₃)₃ | CH₃ |
| A-48 | (S)CH(CH₃)—C(CH₃)₃ | CH₂CH₃ |
| A-49 | (R)CH(CH₃)—C(CH₃)₃ | H |
| A-50 | (R)CH(CH₃)—C(CH₃)₃ | CH₃ |
| A-51 | (R)CH(CH₃)—C(CH₃)₃ | CH₂CH₃ |
| A-52 | (±)CH(CH₃)—CF₃ | H |
| A-53 | (±)CH(CH₃)—CF₃ | CH₃ |
| A-54 | (±)CH(CH₃)—CF₃ | CH₂CH₃ |
| A-55 | (S)CH(CH₃)—CF₃ | H |
| A-56 | (S)CH(CH₃)—CF₃ | CH₃ |
| A-57 | (S)CH(CH₃)—CF₃ | CH₂CH₃ |
| A-58 | (R)CH(CH₃)—CF₃ | H |
| A-59 | (R)CH(CH₃)—CF₃ | CH₃ |
| A-60 | (R)CH(CH₃)—CF₃ | CH₂CH₃ |
| A-61 | (±)CH(CH₃)—CCl₃ | H |
| A-62 | (±)CH(CH₃)—CCl₃ | CH₃ |
| A-63 | (±)CH(CH₃)—CCl₃ | CH₂CH₃ |
| A-64 | (S)CH(CH₃)—CCl₃ | H |
| A-65 | (S)CH(CH₃)—CCl₃ | CH₃ |
| A-66 | (S)CH(CH₃)—CCl₃ | CH₂CH₃ |
| A-67 | (R)CH(CH₃)—CCl₃ | H |
| A-68 | (R)CH(CH₃)—CCl₃ | CH₃ |
| A-69 | (R)CH(CH₃)—CCl₃ | CH₂CH₃ |
| A-70 | CH₂CF₂CF₃ | H |
| A-71 | CH₂CF₂CF₃ | CH₃ |
| A-72 | CH₂CF₂CF₃ | CH₂CH₃ |
| A-73 | CH₂(CF₂)₂CF₃ | H |
| A-74 | CH₂(CF₂)₂CF₃ | CH₃ |
| A-75 | CH₂(CF₂)₂CF₃ | CH₂CH₃ |
| A-76 | CH₂C(CH₃)═CH₂ | H |
| A-77 | CH₂C(CH₃)═CH₂ | CH₃ |
| A-78 | CH₂C(CH₃)═CH₂ | CH₂CH₃ |
| A-79 | CH₂CH═CH₂ | H |
| A-80 | CH₂CH═CH₂ | CH₃ |
| A-81 | CH₂CH═CH₂ | CH₂CH₃ |
| A-82 | CH(CH₃)CH═CH₂ | H |
| A-83 | CH(CH₃)CH═CH₂ | CH₃ |
| A-84 | CH(CH₃)CH═CH₂ | CH₂CH₃ |
| A-85 | CH(CH₃)C(CH₃)═CH₂ | H |
| A-86 | CH(CH₃)C(CH₃)═CH₂ | CH₃ |
| A-87 | CH(CH₃)C(CH₃)═CH₂ | CH₂CH₃ |
| A-88 | CH₂—C≡CH | H |
| A-89 | CH₂—C≡CH | CH₃ |
| A-90 | CH₂—C≡CH | CH₂CH₃ |
| A-91 | cyclopentyl | H |
| A-92 | cyclopentyl | CH₃ |
| A-93 | cyclopentyl | CH₂CH₃ |
| A-94 | cyclohexyl | H |
| A-95 | cyclohexyl | CH₃ |
| A-96 | cyclohexyl | CH₂CH₃ |
| A-97 | CH₂—C₆H₅ | H |
| A-98 | CH₂—C₆H₅ | CH₃ |
| A-99 | CH₂—C₆H₅ | CH₂CH₃ |

| | |
|---|---|
| A-100 | —(CH₂)₂CH═CHCH₂— |
| A-101 | —(CH₂)₂C(CH₃)═CHCH₂— |
| A-102 | —CH(CH₃)CH₂—CH═CHCH₂— |
| A-103 | —(CH₂)₃CHFCH₂— |
| A-104 | —(CH₂)₂CHF(CH₂)₂— |
| A-105 | —CH₂CHF(CH₂)₃— |
| A-106 | —(CH₂)₂CH(CF₃)(CH₂)₂— |
| A-107 | —(CH₂)₂O(CH₂)₂— |
| A-108 | —(CH₂)₂S(CH₂)₂— |
| A-109 | —(CH₂)₄— |
| A-110 | —CH₂CH═CHCH₂— |
| A-111 | —CH(CH₃)(CH₂)₃— |
| A-112 | —CH₂CH(CH₃)(CH₂)₂— |
| A-113 | —CH(CH₃)—(CH₂)₂—CH(CH₃)— |
| A-114 | —CH(CH₃)—(CH₂)₄— |
| A-115 | —CH₂—CH(CH₃)—(CH₂)₃— |
| A-116 | —(CH₂)—CH(CH₃)—CH₂—CH(CH₃)—CH₂— |
| A-117 | —CH(CH₂CH₃)—(CH₂)₄— |
| A-118 | —(CH₂)₆— |
| A-119 | —CH(CH₃)—(CH₂)₅— |
| A-120 | —(CH₂)₂—N(CH₃)—(CH₂)₂— |
| A-121 | —N═CH—CH═CH— |
| A-122 | —N═C(CH₃)—CH═C(CH₃)— |
| A-123 | —N═C(CF₃)—CH═C(CF₃)— |
| A-124 | —(CH₂)₂CH(CH₃)(CH₂)₂— |
| A-125 | —(CH₂)₅— |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phakopsora pachyrhizi and P. meibomiae on soya,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necatoron grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg of seed, preferably 1 to 200 g/100 kg, in particular 5 to 100 g/100 kg are generally used.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

1. Products for Dilution with Water

### A Water-Soluble Concentrates (SL)

10 parts by weight of a compound according to the invention are dissolved in water or in a water-soluble solvent. As an alternative, wetting agents or other auxiliaries are added. The active compound dissolves upon dilution with water.

### B Dispersible Concentrates (DC)

20 parts by weight of a compound according to the invention are dissolved in cyclohexanone with addition of a dispersant, for example polyvinylpyrrolidone. Dilution with water gives a dispersion.

### C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion.

### D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

### E Suspensions (SC, OD)

In an agitated ball mill, 20 parts by weight of a compound according to the invention are comminuted with addition of dispersants, wetting agents and water or an organic solvent to give a fine active compound suspension. Dilution with water gives a stable suspension of the active compound.

### F Water-Dispersible Granules and Water-Soluble Granules (WG, SG)

50 parts by weight of a compound according to the invention are ground finely with addition of dispersants and wetting agents and made into water-dispersible or water-soluble granules by means of technical appliances (for example extrusion, spray tower, fluidized bed). Dilution with water gives a stable dispersion or solution of the active compound.

### G Water-Dispersible Powders and Water-Soluble Powders (WP, SP)

75 parts by weight of a compound according to the invention are ground in a rotor-stator mill with addition of dispersants, wetting agents and silica gel. Dilution with water gives a stable dispersion or solution of the active compound.

### 2. Products to be Applied Undiluted

### H Dustable Powders (DP)

5 parts by weight of a compound according to the invention are ground finely and mixed intimately with 95% of finely divided kaolin. This gives a dustable product.

### I Granules (GR, FG, GG, MG)

0.5 part by weight of a compound according to the invention is ground finely and combined with 95.5% carriers. Current methods are extrusion, spray-drying or the fluidized bed. This gives granules to be applied undiluted.

### J ULV Solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. Alternatively, it is possible to prepare concentrates composed of active substance, wetting agent, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetting agents, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, enilconazole, epoxiconazole, fenbuconazole, fluquinconazole, flusilazole, flutriafol, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mandipropamid, mepronil, nuarimol, penthiopyrad, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phosphorous acid, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

With appropriate modification of the starting materials, the procedures given in the synthesis examples below were used to obtain further compounds I. The compounds obtained in this manner are listed in the table below, together with physical data.

### Example 1

### Preparation of 5-methoxy-6-(2-chlorophenyl)-7-(2-methylpiperidin-1-yl)-1,2,4-triazolo[1,5a]pyrimidine

### Step 1: 5-Chloro-6-(2-chlorophenyl)-7-(2-methylpiperidin-1-yl)-1,2,4-triazolo[1,5a]-pyrimidine

A solution of 1.5 g (5 mmol) of 5,7-dichloro-6-(2-chlorophenyl)-1,2,4-triazolo[1,5a]-pyrimidine (cf. WO 03/80615), 0.5 g (5.1 mmol) of triethylamine and 0.5 g (5.1 mmol) of 2-methylpiperidine in 10 ml of methylene chloride were stirred at 20-25° C. for about 15 hours. After addition of 0.05 g (0.51 mmol) of triethylamine and 0.05 g (0.51 mmol) of 2-methylpiperidine, the mixture was then stirred at 20-25° C. for a further 20 hours. The reaction mixture was then extracted with dil. hydrochloric acid and water. After drying, the solvent was removed from the organic phase. This gave 1.5 g of 5-chloro-6-(2-chlorophenyl)-7-(2-methylpiperidin-1-yl)-1,2,4-triazolo[1,5a]pyrimidine (purity (HPLC): 64%) as a yellow oil which was used for the next reaction without further purification. ¹H-NMR (CDCl₃, 6 in ppm): 2 rotamers about 2:1: 8.4 (2s, 1H); 7.55 (m, 1H); 7.2-7.5 (m, 3H); 4.6; 4.15 (2m, 1H); 3.55; 3.1 (2m, 1H); 3.35; 2.7 (2m, 1H); 1.25 to 1.9 (m, 6H); 1.15 (2d, 3H).

### Step 2: 5-Methoxy-6-(2-chlorophenyl)-7-(2-methylpiperidin-1-yl)-1,2,4-triazolo[1,5a]-pyrimidine [I-1]

1.5 g of the compound from ex. 1 (purity about 64%, about 2.7 mmol) and 1.2 g of a 30% strength sodium methoxide solution (6.7 mmol of sodium methoxide) in 20 ml of methanol were stirred at 20-25° C. for about 15 hours. The reaction mixture was then diluted with water and the aqueous phase was extracted with methylene chloride. The combined aqueous phases were washed with water and dried, and the solvent was removed. The residue was purified by preparative MPLC on silica gel RP-18. This gave 0.4 g of the title compound as a colorless solid of m.p. 186-187° C.).

¹H-NMR (CDCl₃, δ in ppm): 2 rotamers about 3:1: 8.2 (s, broad, 1H); 7.5 (m, 1H); 7.2-7.4 (m, 3H); 4.5; 3.9 (2m, 1H); 4.0 (s, 3H); 3.4; 2.9 (2m, 1H); 3.3; 2.75 (2m, 1H); 1.15-1.9 (m, 6H); 1.15; 1.05 (2d, 3H).

TABLE 1

| | | | | | Phys. data (m.p. [° C.], ¹H-NMR |
|---|---|---|---|---|---|
| No. | R¹ | R² | L | X | [δ: ppm {CDCl₃}]) |
| I-1 | —CH(CH₃)—(CH₂)₄— | | Cl | OCH₃ | 186-187 |
| I-2 | —(CH₂)₂—CH(CH₃)—(CH₂)₂— | | Cl | CH₃ | 207-208 |
| I-3 | —CH(CH₃)—(CH₂)₄— | | F | CN | 201-202 |
| I-4 | —CH(CH₃)—(CH₂)₄— | | F | OCH₃ | 170-172 |
| I-5 | —CH(CH₃)—(CH₂)₄— | | F | CH₃ | 160-164 |
| I-6 | —CH(CH₃)—(CH₂)₄— | | Cl | CH₃ | 184-186 |
| I-7 | (S) CH(CH₃)—CF₃ | H | Cl | CH₃ | 8.35 (s, 1H); 7.65 (m, 1H); |
| | | | | | 7.5 (m, 2H); 7.35 (m, 1H); 2.3, |
| | | | | | 2.25 (2s, 3H); 1.3, 1.25 (2d, 3H) |
| I-8 | (S) CH(CH₃)—CF₃ | H | F | CH₃ | 149-151 |
| I-9 | —CH(CH₃)—(CH₂)₄— | | CH₃ | CN | 176-178 |
| I-10 | —CH(CH₃)—(CH₂)₄— | | CH₃ | OCH₃ | 211-213 |
| I-11 | —CH(CH₃)—(CH₂)₄— | | CH₃ | CH₃ | 182-184 |
| I-12 | (S) CH(CH₃)—CF₃ | H | CH₃ | CH₃ | 128-130 |

| | | | | |
|---|---|---|---|---|
| I-13 | —(CH₂)₂—CH(CH₃)—(CH₂)₂— | Cl | OC₂H₅ | 8.3 (s, 1H); 7.5 (m, 1H); |
| | | | | 7.35 (m, 2H); 7.2 (m, 1H); 4.5 (m, |
| | | | | 1H); 4.4 (m, 1H); 0.95 (d, 3H) |
| I-14 | —(CH₂)₂—CH(CH₃)—(CH₂)₂— | Cl | OCH₃ | 209-211 |

### Examples for the Action Against Harmful Fungi

The fungicidal action of the compounds of the formula I was demonstrated by the following tests:

The active compounds were prepared separately as a stock solution comprising 25 mg of active compound which was made up to 10 ml using a mixture of acetone and/or DMSO and the emulsifier Uniperol® EL (wetting agent having an emulsifying and dispersing action based on ethoxylated alkylphenols) in a ratio by volume of solvent/emulsifier of 99:1. The mixture was then made up to 100 ml with water. This stock solution was diluted with the solvent/emulsifier/water mixture described to give the concentration of active compound stated below. Alternatively, the active compounds were employed as a commercial finished formulation and diluted with water to the stated concentration of active compound.

### Use Example 1

### Activity Against Early Blight of Tomato Caused by Alternaria solani

Leaves of potted plants of the cultivar “Goldene Königin” were sprayed to runoff point with an aqueous suspension having the active compound concentration stated below. The next day, the leaves were infected with an aqueous spore suspension of Alternaia solaniin a 2% biomalt solution having a density of 0.17×10⁶ spores/ml. The plants were then placed in a water vapor-saturated chamber at temperatures between 20 and 22° C. After 5 days, the disease on the untreated but infected control plants had developed to such an extent that the infection could be determined visually in %.

In this test, the plants which had been treated with 250 ppm of the compound I-2, I-5 to I-9, I-11, I-12 or I-14 showed an infection of not more than 5%, whereas the untreated plants were 90% infected.

### Use Example 2

### Activity Against Gray Mold on Bell Pepper Leaves Caused by Botrytis cinerea, Protective Application

Bell pepper seedlings of the cultivar “Neusiedler Ideal Elite” were, after 2-3 leaves were well developed, sprayed to runoff point with an aqueous suspension having the active compound concentration stated below. The next day, the treated plants were inoculated with a spore suspension of Botrytis cinerea which comprised 1.7×10⁶ spores/ml in a 2% strength aqueous biomalt solution. The test plants were then placed in a dark climatized chamber at 22 to 24° C. and high atmospheric humidity. After 5 days, the extent of the fungal infection on the leaves could be determined visually in %.

In this test, the plants which had been treated with 250 ppm of the compound I-2, I-5 to I-9, I-11, I-12, I-13 or I-14 showed an infection of not more than 20%, whereas the untreated plants were 100% infected.

1. A triazolopyrimidine of the formula I 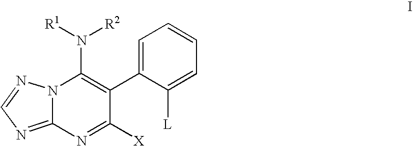
in which the substituents are as defined below:
R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle comprising one to four heteroatoms from the group consisting of O, N and S,
R² is hydrogen or one of the groups mentioned under R¹,
R¹ and R² together with the nitrogen atom, to which they are attached, may also form a five- or six-membered heterocyclyl or heteroaryl which is attached via N and which may comprise one to three further heteroatoms from the group consisting of O, N and S as ring member and carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;
R¹ and/or R² may carry one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkyleneoxy, phenyl, naphthyl, a five- to ten-membered saturated, partially unsaturated or aromatic heterocycle comprising one to four heteroatoms from the group consisting of O, N and S,
where these aliphatic, alicyclic or aromatic groups for their part may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals comprise 1 to 6 carbon atoms and the alkenyl or alkynyl groups mentioned in these radicals comprise 2 to 8 carbon atoms;
and/or one to three of the following radicals:
cycloalkyl, cycloalkoxy, heterocyclyl, heterocyclyloxy, where the cyclic systems comprise 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably comprise 6 to 10 ring members and the hetaryl radicals comprise 5 or 6 ring members, where the cyclic systems may be partially or fully halogenated or substituted by alkyl or haloalkyl groups;
L is fluorine, chlorine or methyl,
X is cyano, C₁-C₄-alkyl, C₁-C₄-alkoxy or C₁-C₂-haloalkoxy;
where X is not methyl if R¹ and R² together are n-pentylene or 3-methyl-n-pentylene and L is fluorine, or R¹ and R² together are 3-methyl-n-pentylene and L is chlorine.

2. The compound of the formula I according to claim 1, with the proviso that R¹ and R² together may not be piperidin-1-yl or 4-alkylpiperidin-1-yl.

3. The compound of the formula I according to claim 1 in which X is cyano, C₁-C₄-alkoxy or C₁-C₂-haloalkoxy.

4. The compound of the formula I according to claim 1 in which X is C₁-C₄-alkyl.

5. The compound of the formula I according to any of claim 1 in which L is methyl.

6. The compound of the formula I according to any of claim 1 in which R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-halogenalkynyl oder phenyl, naphthyl, or a five- or six-membered saturated, partially unsaturated or aromatic heterocycle comprising one to four heteroatoms from the group consisting of O, N and S and R² is hydrogen or C₁-C₄-alkyl, or
together with the nitrogen atom, to which they are attached, form a five- or six-membered heterocyclyl or heteroaryl which is attached via N and which may comprise one to three further heteroatoms from the groups consisting of O, N and S as ring member, where R¹ and R² may be substituted according to claim 1.

7. The compound of the formula I according to any of claim 1 in which R² is not hydrogen.

8. The compound of the formula I according to claim 1 corresponding to the formula I.1: 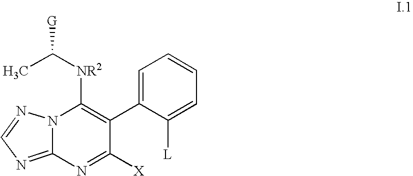
in which G is C₂-C₆-alkyl, C₁-C₄-alkoxymethyl or C₃-C₆-cycloalkyl.

9. The compound of the formula I according to claim 1 corresponding to the formula I.2: 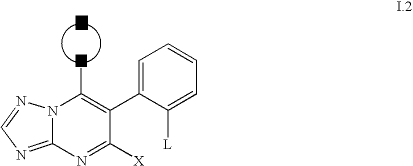
in which D together with the nitrogen atom form a five- or six-membered heterocyclyl or heteroaryl which is attached via N and may comprise a further heteroatom from the group consisting of O, N and S as ring member and/or may carry one or more substituents from the group consisting of halogen, C₁-C₄-alkyl, C₁-C₄-alkoxy and C₁-C₂-haloalkyl.

10. A process for preparing compounds of the formula I according to claim 1 in which X is cyano, C₁-C₄-alkyl, C₁-C₄-alkoxy or C₁-C₂-haloalkoxy by reaction of 5-amino-triazole of the formula II 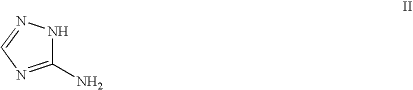
with phenylmalonates of the formula II 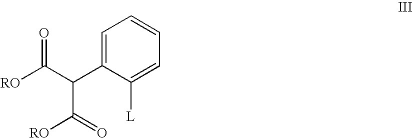
with pyrimidines of the formula IV, 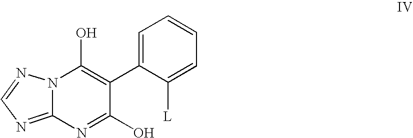
halogenation to give the dihalo compounds of the formula V 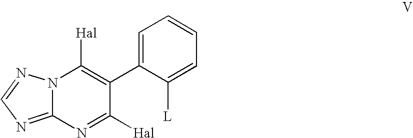
and reaction of V with amines of the formula VI 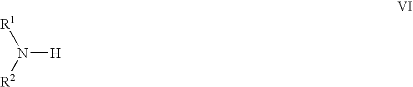
to give compounds of the formula VII, 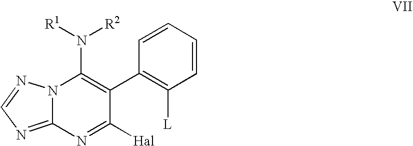
reaction of compounds VII with compounds of the formula VIII
M-X′ VIII
which, depending on the group X′ to be introduced, are inorganic cyanides, alkoxides or haloalkoxides and in which M is an ammonium, tetraalkylammonium, alkali metal or alkaline earth metal cation, and, if desired, for preparing compounds of the formula I as claimed in claim 1 in which X is alkyl by reaction of the compounds VII with malonates of the formula IX 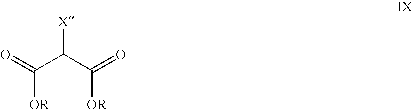
in which X″ is hydrogen or C₁-C₃-alkyl and R is C₁-C₄-alkyl to give compounds of the formula X 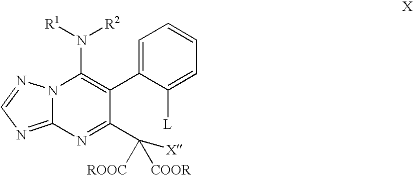
and decarboxylation to give compounds I in which X is alkyl.

11. A process for preparing compounds of the formula I according to claim 1 in which X is C₁-C₄-alkyl or C₁-C₄-haloalkyl by reaction of 5-aminotriazole of the formula II according to claim 2 with keto esters of the formula IIIa 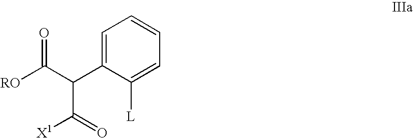
in which X¹ is C₁-C₄-alkyl or C₁-C₄-haloalkyl and R is C₁-C₄-alkyl to give 5-alkyl-7-hydroxy-6-phenyltriazolopyrimidines of the formula IVa, 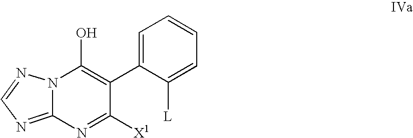
halogenation of IVa to give 7-halotriazolopyrimidines of the formula Va 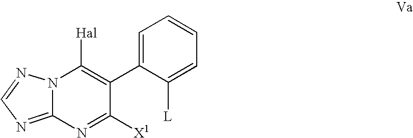
and reaction of Va with amines of the formula VI according to claim 2 to give compounds I in which X is C₁-C₄-alkyl or C₁-C₄-haloalkyl.

12. A fungicidal composition comprising a solid or liquid carrier and a compound of the formula I according to claim 1.

13. Seed comprising from 1 to 1000 g of a compound of the formula I according to claim 1 per 100 kg.

14. A method for controlling phytopathogenic harmful fungi, which method comprises treating the fungi or the materials, plants, the soil or seed to be protected against fungal attack with an effective amount of a compound of the formula I according to claim 1.

15. The compound of the formula I according to claim 2 in which L is methyl.

16. The compound of the formula I according to claim 3 in which L is methyl.

17. The compound of the formula I according to claim 4 in which L is methyl.

18. The compound of the formula I according to claim 2 in which R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-halogenalkynyl oder phenyl, naphthyl, or a five- or six-membered saturated, partially unsaturated or aromatic heterocycle comprising one to four heteroatoms from the group consisting of O, N and S and R² is hydrogen or C₁-C₄-alkyl, or
together with the nitrogen atom, to which they are attached, form a five- or six-membered heterocyclyl or heteroaryl which is attached via N and which may comprise one to three further heteroatoms from the groups consisting of O, N and S as ring member, where R¹ and R² may be substituted.

19. The compound of the formula I according claim 2 in which R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-halogenalkynyl oder phenyl, naphthyl, or a five- or six-membered saturated, partially unsaturated or aromatic heterocycle comprising one to four heteroatoms from the group consisting of O, N and S and R² is hydrogen or C₁-C₄-alkyl, or
together with the nitrogen atom, to which they are attached, form a five- or six-membered heterocyclyl or heteroaryl which is attached via N and which may comprise one to three further heteroatoms from the groups consisting of O, N and S as ring member, where R¹ and R² may be substituted.

20. The compound of the formula I according claim 3 in which R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-halogenalkynyl oder phenyl, naphthyl, or a five- or six-membered saturated, partially unsaturated or aromatic heterocycle comprising one to four heteroatoms from the group consisting of O, N and S and R² is hydrogen or C₁-C₄-alkyl, or
together with the nitrogen atom, to which they are attached, form a five- or six-membered heterocyclyl or heteroaryl which is attached via N and which may comprise one to three further heteroatoms from the groups consisting of O, N and S as ring member, where R¹ and R² may be substituted.
